(19) United States
(12) Patent Application Publication
(10) Pub. No.: US 2011/0237665 A1
(43) Pub. Date: Sep. 29, 2011

(54) Agrochemical Composition Comprising Pesticide and UV Absorber

(75) Inventors: Andrea Misske, Speyer (DE); Christian Bittner, Bensheim (DE); Michael Ishaque, Mannheim (DE); Markus Hoffmann, Barcelona (ES); Richard Riggs, Mannheim (DE); Sens Ruediger, Ludwigshafen (DE); Andre Kabat, Hassloch (DE); Sylke Haremza, Neckargemuend (DE); Douglas D. Anspaugh, Apex, NC (US); David M. Terry, Cary, NC (US)

(73) Assignee: BASF SE, Ludwigshafen (DE)

(21) Appl. No.: 13/132,254

(22) PCT Filed: Nov. 30, 2009
(86) PCT No.: PCT/EP2009/066009

§ 371 Date: Jun. 1, 2011

Related U.S. Application Data
(60) Provisional application No. 61/119,556, filed on Dec. 3, 2008.

(51) Int. Cl. A01N 47/34; A01N 53/08; C07C 255/44; C07D 251/54; C07C 219/22; A01P 7/04; A01P 3/00; A01P 13/00

(52) U.S. Cl. 514/522; 514/521; 558/402; 544/197; 560/52

(57) ABSTRACT

The present invention relates to an agrochemical composition comprising pesticide and UV absorber, and the abovementioned UV absorbers. The invention furthermore relates to the use of the UV absorbers in agrochemical compositions. It moreover relates to a method of controlling phytopathogenic fungi and/or undesirable plant growth and/or undesirable insect or mite infestation and/or of regulating plant growth.

The present invention relates to an agrochemical composition comprising pesticide and UV absorber, and the abovementioned UV absorbers. The invention furthermore relates to the use of the UV absorbers in agrochemical compositions. It moreover relates to a method of controlling phytopathogenic fungi and/or undesirable plant growth and/or undesirable insect or mite infestation and/or of regulating plant growth. Combinations of preferred features with other preferred features are comprised in the present invention.

Agrochemical compositions comprising pesticide and UV absorber are generally known:

WO 1992/03926 discloses insecticidal compositions comprising a pyrethroid, a UV absorption agent and an antioxidant.

EP 0 376 888 A1 discloses compositions for controlling harmful insects comprising a substance which modifies the behavior of the harmful insects and a pesticidally active compound, both of which are comprised in a flowable matrix which protect the behavior-modifying substance from UV radiation. The compositions in general comprise from 51 to 98 wt. % of the matrix, which is conventionally a UV absorber which preferably has a viscosity of from 1,000 to 40,000 cp. A suitable UV absorber is, for example, a mixture of the following alkoxylated 2-(2-hydroxyphenyl)-benzotriazoles TinuA and TinuB

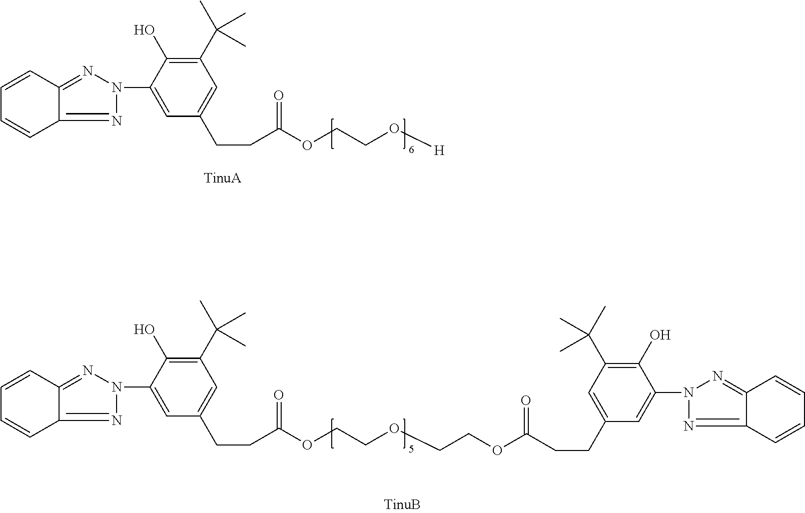

in a weight ratio of TinuA to TinuB of 50 to 38, which is obtainable under the trade name Tinuvin® 1130 from Ciba.

WO 2006/089747 discloses a method for protecting materials comprising the use of a composition comprising a capsule which contains a photolabile pesticide and a UV absorber such as alpha-[3-[3-(2H-benzotriazol-2-yl)-5-(1,1-dimethylethyl)-4-hydroxyphenyl]-1-oxopropyl]-omega-hydroxy-poly(oxy-1,2-ethanediyl). WO 1997/42815 discloses a composition comprising 0.1 to 20% of pesticide, 0.01 to 30% of pheromone and 40 to 98% of UV absorber. Tinuvin 1130, for example, can be employed as the UV absorber.

WO 2008/085682 discloses a composition comprising photolabile pesticide and a UV stabilizer, which can be, for example, Uvinul® P25 (PEG-25 para-aminobenzoic acid).

Cinnamic acid derivatives which comprise alkoxylated groups are likewise known: Philippon et al. (Synthetic. Communications, 1997, 27(15), 2651-2682) disclose a compound of the formula

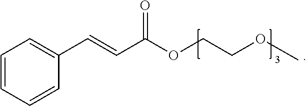

The UV absorbers from the prior art have various disadvantages: Further auxiliaries, such as antioxidants, must be added. The UV absorbers must be employed in a matrix. The UV absorbers have not been able to stabilize light-sensitive pesticides for a sufficiently long period of time.

The object of the present invention was to provide alternative UV absorbers for use in agrochemical compositions. A further object was to discover UV absorbers which stabilize UV-sensitive pesticides. The stabilization should be better than in the prior art, in particular also at relatively low concentrations of UV absorber. An object was furthermore to discover UV absorbers which allow simpler formulation of pesticides, for example in that fewer auxiliaries, such as surfactants, are necessary.

The object was achieved by an agrochemical composition comprising pesticide and UV absorber, wherein the UV absorber corresponds to the formula I

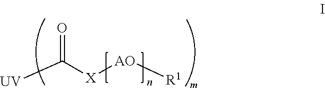

wherein AO: C₂-C₄ alkoxy or CH₂CH₂NH;
   n: 3-50;
   m: 1 if UV represents A or B, 3 if UV represents C;
   X: NH or O;
   R¹: H or C₁-C₂₄ alkyl;
   UV: is a group chosen from the formulae A to C

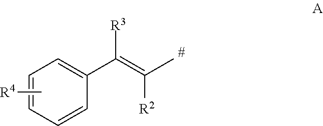

wherein R²: H, CN or CO₂—(C₁-C₁₆ alkyl);
   R³: H, C₁-C₆ alkyl, phenyl or phenyl substituted by C₁-C₁₈ alkoxy;
   R⁴: H or C₁-C₁₈ alkoxy;

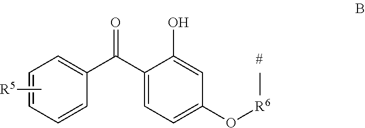

wherein R⁵: H, C₁-C₁₈ alkyl, OH, or C₁-C₁₈ alkoxy;
   R⁶: C₁-C₈ alkylene; or

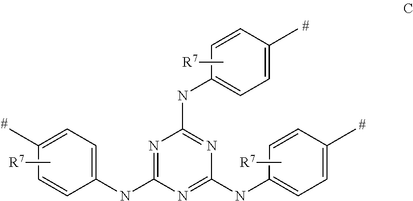

wherein R⁷: H or C₁-C₁₂ alkyl;
which are bonded via # to the carbonyl group of I.

The term pesticide designates at least one active compound chosen from the group of fungicides, insecticides, nematicides, herbicides, safeners and/or growth regulators. Preferred pesticides are fungicides, insecticides and herbicides, in particular insecticides. Mixtures of pesticides from two or more of the abovementioned classes can also be used. The person skilled in the art is familiar with such pesticides, which can be found, for example, in Pesticide Manual, 14th Ed. (2006), The British Crop Protection Council, London. Suitable insecticides are insecticides from the class of carbamates, organophosphates, organochlorine insecticides, phenylpyrazoles, pyrethroids, neonicotinoids, spinosins, avermectins, milbemycins, juvenile hormone analogues, alkyl halides, organotin compounds, nereistoxin analogues, benzoylureas, diacylhydrazines, METI acaricides, and insecticides such as chloropicrin, pymetrozine, flonicamid, clofentezine, hexythiazox, etoxazole, diafenthiuron, propargite, tetradifon, chlorfenapyr, DNOC, buprofezin, cyromazine, amitraz, hydramethylnon, acequinocyl, fluacrypyrim, rotenone, or derivatives thereof. Suitable fungicides are fungicides of the classes dinitroanilines, allylamines, anilinopyrimidines, antibiotics, aromatic hydrocarbons, benzenesulfonamides, benzimidazoles, benzisothiazoles, benzophenones, benzothiadiazoles, benzotriazines, benzylcarbamates, carbamates, carboxamides, carboxylic acid amides, chloronitriles, cyanoacetamide oximes, cyanoimidazoles, cyclopropanecarboxamides, dicarboximides, dihydrodioxazines, dinitrophenylcrotonates, dithiocarbamates, dithiolanes, ethylphosphonates, ethylaminothiazolecarboxamides, guanidines, hydroxy-(2-amino-)pyrimidines, hydroxyanilides, imidazoles, imidazolinones, inorganic compounds, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, thiazolecarboxamides, thiocarbamates, thiocarbamates, thiophanates, thiophenecarboxamides, toluamides, triphenyltin compounds, triazines, triazoles. Suitable herbicides are herbicides of the classes of acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolinones, triazolocarboxamides, triazolopyrimidines, triketones, uracils, ureas.

Preferred herbicides are napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norfiurazon, thiazopyr, triclopyr, dithiopyr, diflufenican, picolinafen, amidosulfuron, molinate, vernolate, prometon , metribuzin, azafenidin, carfentrazone-ethyl, sulfentrazone, metoxuron, monolinuron, fluchloralin and flurenol. Preferred fungicides are cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichiofluamid, bromuconazole and myclobutanil. Preferred insecticides are acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon. In a further embodiment, preferred insecticides are pyrethroids or metaflumizone, in particular pyrethroids. Particularly preferred pesticides are alpha-cypermethrin and metaflumizone.

In one embodiment, pesticides which are UV-sensitive are used. This UV sensitivity can be determined in simple preliminary tests. Pesticides are preferably regarded as UV-sensitive if on irradiation with UV light, preferably UV/VIS light of wavelength 300-800 nm, of a pesticide film which by drying of a 25 wt. % solution of the pesticide in a suitable solvent, preferably in acetone, they are degraded to the extent of at least 20 wt. % within 24 h at 25° C. The illuminance used is typically in the range from 10 000 to 100 000 lux and preferably in the range from 50 000 to 80 000 lux. The pesticides are deemed degraded when the concentration of the pesticide (or of one pesticidal component in a mixture of two or more pesticidal components) is correspondingly reduced.

Suitable UV absorbers are the structures of the formula I as defined above. Suitable radicals AO are C₂-C₄ alkoxy or —CH₂CH₂NH—, preferably —CH₂CH₂NH—, wherein C₂-C₄ alkoxy is preferably —CH₂CH₂O—, —CH(CH₃)CH₂O— or —CH(CH₂CH₃)CH₂O—, in particular —CH₂CH₂O—. The index n represents 3 to 50, preferably 3 to 30, in particular 3 to 25 and specifically 3 to 15. The index m represents 1 if UV represents A, B or D, or represents 3 if UV represents C. Suitable radicals X are NH or O, preferably O.

Suitable radicals R¹ are H or C₁-C₂₄ alkyl, preferably C₁-C₆ alkyl, in particular CH₃. In a further embodiment, R₁ represents H or C₁-C₂₄ alkyl, preferably C₆-C₂₄ alkyl, particularly preferably C₆-C₂₄ alkyl, in particular C₈-C₂₀ alkyl, specifically C₁₀-C₂₀ alkyl, very specifically C₁₀-C₁₀ branched alkyl.

The expression “—CH₂CH₂NH—” is a general empirical formula and represents a monomer unit of a polyethyleneimine group. These polyethyleneimine groups can be linear or branched, and they are preferably branched. Branched polyethyleneimine groups conventionally contain primary, secondary and tertiary amino groups. The molar ratio of primary/secondary/tertiary amino groups can be in the range of from 1/0.5/0.2 to 1/1.9/1.5, preferably in the range of from 1/0.7/0.4 to 1/1.5/1.1.

Suitable groups UV are groups chosen from the formulae A to C, preferably from B or C, wherein the groups are bonded via # to the carbonyl group of the formula I.

A suitable radical UV is a structure of the formula A

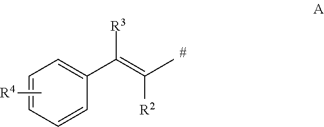

wherein R²: H, CN or CO₂—(C₁-C₁₆ alkyl), preferably CN or CO₂—(C₁-C₁₆ alkyl), in particular CN;
   R³: H, C₁-C₆ alkyl, phenyl or phenyl substituted by C₁-C₁₈ alkoxy, preferably C₁-C₆ alkyl, phenyl or phenyl substituted by C₁-C₁₈ alkoxy, in particular phenyl or phenyl substituted by C₁-C₁₈ alkoxy; and
   R⁴: H or C₁-C₁₈ alkoxy, preferably H.

A further suitable radical UV is a structure of the formula B

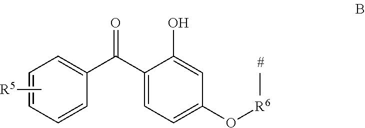

wherein R⁵: H, C₁-C₁₈ alkyl, OH or C₁-C₁₈ alkoxy, preferably H; and
   R⁶: C₁-C₈ alkylene, preferably C₁-C₂ alkylene, in particular CH₂.

A further suitable radical UV is a structure of the formula C

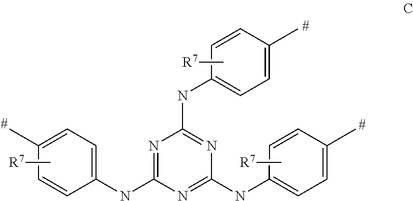

wherein R⁷: H or C₁-C₁₂ alkyl, preferably H.

In a preferred embodiment, the agrochemical composition comprises pesticide and UV absorber, wherein the UV absorber corresponds to a structure of the formula III

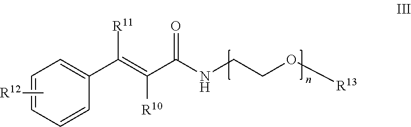

wherein
n: 3 to 50, preferably 3 to 25;
R¹⁰: H, CN or CO₂—(C₁-C₁₆ alkyl), preferably H or CN, in particular H;
R¹¹: H, C₁-C₆ alkyl, phenyl or phenyl substituted by C₁-C₁₈ alkoxy, preferably H, phenyl or phenyl substituted by C₁-C₁₈ alkoxy, in particular H;
R¹²: H or C₁-C₁₈ alkoxy, preferably H; and
R¹³: H or C₁-C₂₄ alkyl, preferably CH₃.

In a further preferred embodiment, R¹³ represents H or C₁-C₂₄ alkyl, preferably C₆-C₂₄ alkyl, particularly preferably C₆-C₂₄ alkyl, in particular C₈-C₂₀ alkyl, specifically C₁₀-C₂₀ alkyl, very specifically C₁₀-C₂₀ branched alkyl.

In a further preferred embodiment, the agrochemical composition comprises pesticide and UV absorber, wherein the UV absorber corresponds to a structure of the formula IV

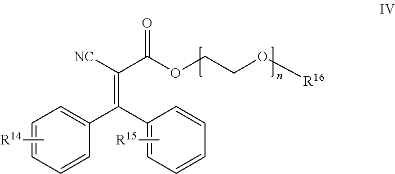

wherein
n: 3 to 50, preferably 3 to 25;
R¹⁴, R¹⁵: independently of one another H or C₁-C₁₈ alkoxy, preferably H or methoxy, in particular H; and
R¹⁶: H or C₁-C₂₄ alkyl, preferably C₁-C₄ alkyl, particularly preferably CH₃.

In a further preferred embodiment, R¹⁶ represents H or C₁-C₂₄ alkyl, preferably C₆-C₂₄ alkyl, particularly preferably C₆-C₂₄ alkyl, in particular C₈-C₂₀ alkyl, specifically C₁₀-C₂₀ alkyl, very specifically C₁₀-C₂₀ branched alkyl.

In a further preferred embodiment, the agrochemical composition comprises pesticide and UV absorber, wherein the UV absorber corresponds to a structure of the formula V

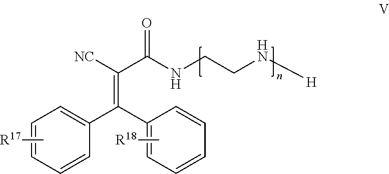

wherein
n: 3 to 50, preferably 3 to 25;
R¹⁷, R¹⁸: independently of one another H or C₁-C₁₈ alkoxy, preferably H or methoxy, in particular H.

In a further preferred embodiment, the agrochemical composition comprises pesticide and UV absorber, wherein the UV absorber corresponds to a structure of the formula VI

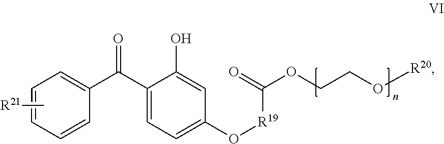

wherein
n: 3 to 50, preferably 3 to 20, in particular 3 to 10;
R¹⁹: C₁-C₈ alkylene, preferably C₁-C₄ alkylene, in particular CH₂;
R²⁰: H or C₁-C₂₄ alkyl, preferably CH₃; and
R²¹: H, C₁-C₁₈ alkyl, OH, or C₁-C₁₈ alkoxy, preferably H.

In a further preferred embodiment, R²⁰ represents H or C₁-C₂₄ alkyl, preferably C₆-C₂₄ alkyl, particularly preferably C₆-C₂₄ alkyl, in particular C₈-C₂₀ alkyl, specifically C₁₀-C₂₀ alkyl, very specifically C₁₀-C₂₀ branched alkyl.

In a further preferred embodiment, the agrochemical composition comprises pesticide and UV absorber, wherein the UV absorber corresponds to a structure of the formula VII

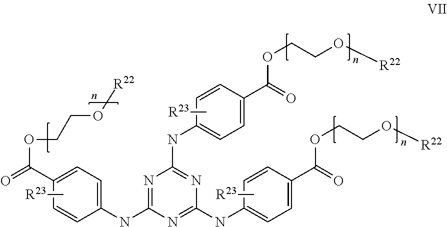

wherein
n: 3 to 50, preferably 3 to 25, in particular 3 to 10;
R²²: H or C₁-C₂₄ alkyl, preferably CH₃; and
R²³: H or C₁-C₁₂ alkyl, preferably H.

In a further preferred embodiment, R²² represents H or C₁-C₂₄ alkyl, preferably C₆-C₂₄ alkyl, particularly preferably C₆-C₂₄ alkyl, in particular C₈-C₂₀ alkyl, specifically C₁₀-C₂₀ alkyl, very specifically C₁₀-C₂₀ branched alkyl.

The agrochemical composition according to the invention in general comprises 0.01 to 95 wt. %, preferably 0.5 to 80 wt. %, particularly preferably 2 to 50 wt. % and specifically 5 to 20 wt. % of pesticide, in each case based on the composition.

The UV absorbers conventionally have a surface tension at the interface of water to air at 25° C. of at most 50 mN/m, preferably of at most 46 mN/m, particularly preferably at most 44 mN/m, specifically at most 40 mN/m.

The agrochemical composition according to the invention in general comprises 0.1 to 50 wt. %, preferably 0.5 to 30 wt. %, particularly preferably 1.0 to 15 wt. % of UV absorber, in each case based on the composition.

The weight ratio of pesticide to UV absorber is usually from 30:1 to 1:2, preferably 15:1 to 2:1, particularly preferably 8:1 to 3:1.

Agrochemical compositions comprising pesticide and UV absorber can be in composition types conventional for agrochemical formulations, e.g. solutions, emulsions, suspensions dusts, powders, pastes and granules. The type depends on the particular intended use; it should in all cases ensure a fine and uniform distribution of the compound according to the invention. Examples of composition types are suspensions (SC, OD, FS), pastes, pastilles, wettable powders or dusts (WP, SP, SS, WS, DP, DS) or granules (GR, FG, GG, MG), which are either soluble or dispersible in water, and gels for treatment of plant propagation materials, such as seed (GF). Baits for animals, such as ants or rats, can be in various of the abovementioned composition types, preferably as powders, pastes, granules or gels. In general the compositions types (e.g. SC, OD, FS, WG, SG, WP, SP, SS, WS, GF) are employed in diluted form. Composition types such as DP, DS, GR, FG, GG, MG or baits are as a rule employed in undiluted form. Preferred composition types are suspensions.

The agrochemical compositions can furthermore also comprise auxiliaries conventional for plant protection compositions, the choice of the auxiliaries depending on the concrete use form or the active compound. Examples of suitable auxiliaries are solvents, solid carrier substances, surface-active substances (such as further solubilizing agents, protective colloids, wetting agents and adhesive agents), organic and inorganic thickeners, bactericides, antifreeze agents, defoamers, optionally dyestuffs and adhesives (e.g. for seed treatment) or conventional auxiliaries for bait formulation (e.g. attractants, feedstuffs, bitters).

Possible solvents are water, organic solvents, such as mineral oil fractions of medium to high boiling point, such as kerosene and diesel oil, furthermore coal tar oils and oils of plant or animal origin, aliphatic, cyclic and aromatic hydrocarbons, e.g. paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl-fatty acid amides, fatty acids and fatty acid esters and strongly polar solvents, e.g. amines, such as N-methylpyrrolidone. In principle, solvent mixtures can also be used, as well as mixture of the abovementioned solvents and water.

Solid carrier substances are mineral earths, such as silicas, silica gels, silicates, talc, kaolin, limestone, lime, chalk, bolus, loam, clay, dolomite, diatomaceous earth, calcium sulfate and magnesium sulfate, magnesium oxide, ground plastics, fertilizers, such as ammonium sulfate, ammonium phosphate, ammonium nitrate, ureas and plant products, such as cereal flour, tree bark, wood and nutshell flour, cellulose powder or other solid carrier substances.

Possible surface-active substances (adjuvants, wetting, adhesive, dispersing or emulsifying agents) are the alkali metal, alkaline earth metal and ammonium salts of aromatic sulfonic acids, e.g. of lignin—(Borresperse® types Borregaard, Norway), phenol-, naphthalene—(Morwet® types, Akzo Nobel, USA) and dibutylnaphthalenesulfonic acid (Nekal® types, BASF, Germany), and of fatty acids, alkyl- and alkylarylsulfonates, alkyl, lauryl ether and fatty alcohol sulfates, and salts of sulfated hexa-, hepta- and octadecanols, as well as of fatty alcohol glycol ethers, condensation products of sulfonated naphthalene and its derivatives with formaldehyde, condensation products of naphthalene or of naphthalenesulfonic acids with phenol and formaldehyde, polyoxyethylene octylphenol ethers, ethoxylated isooctyl-, octyl- or nonylphenol, alkylphenyl or tributylphenyl polyglycol ethers, alkylaryl polyether alcohols, isotridecyl alcohol, fatty alcohol-ethylene oxide condensates, ethoxylated castor oil, polyoxyethylene or polyoxypropylene alkyl ethers, lauryl alcohol polyglycol ether acetate, sorbitol esters, lignin-sulfite waste liquors and proteins, denatured proteins, polysaccharides (e.g. methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and copolymers thereof.

Examples of thickeners (i.e. compounds which impart to the composition modified flow properties, i.e. high viscosity in the resting state and low viscosity in the agitated state) are polysaccharides and organic and inorganic laminar minerals, such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added to stabilize the composition. Examples of bactericides are those based on dichlorophen and benzyl alcohol hemiformal (Proxel® from ICI or Acticide® RS from Thor Chemie and Kathon® MK from Rohm & Haas) and isothiazolinone derivatives, such as alkylisothiazolinones and benzisothiazolinones (Acticide® MBS from Thor Chemie).

Examples of suitable antifreeze agents are ethylene glycol, propylene glycol, urea and glycerol.

Examples of defoamers are silicone emulsions (such as e.g. Silikon® SRE, Wacker, Germany or Rhodorsil®, Rhodia, France), long-chain alcohols, fatty acids, salts of fatty acids, organofluorine compounds and mixtures thereof.

Examples of composition types are:

1. Composition types for dilution in water
i) Water-soluble concentrates (SL, LS)
   10 parts by wt. of the pesticide are dissolved with 90 parts by wt. of water or a water-soluble solvent. Alternatively, wetting agents or other auxiliaries are added. During the dilution in water, the pesticide dissolves. A composition with an active compound content of 10 wt. % is obtained in this manner.
ii) Dispersible concentrates (DC)
   20 parts by wt. of the pesticide are dissolved in 70 parts by wt. of cyclohexanone with the addition of 10 parts by wt. of a dispersing agent, e.g. polyvinylpyrrolidone. On dilution in water, a dispersion results. The active compound content is 20 wt. %
iii) Emulsifiable concentrates (EC)
   15 parts by wt. of the pesticide are dissolved in 75 parts by wt. of xylene with the addition of Ca dodecylbenzenesulfonate and castor oil ethoxylate (in each case 5 parts by wt.). On dilution in water, an emulsion results. The composition has an active compound content of 15 wt. %.
iv) Emulsions (EW, EO, ES)
   25 parts by wt. of the pesticide are dissolved in 35 parts by wt. of xylene with the addition of Ca dodecylbenzenesulfonate and castor oil ethoxylate (in each case 5 parts by wt.). This mixture is added to 30 parts by wt. of water and converted into a homogeneous emulsion by means of an emulsifying machine (e.g. Ultra-Turrax). On dilution in water, an emulsion results. The composition has an active compound content of 25 wt. %.
v) Suspensions (SC, OD, FS)
   20 parts by wt. of the pesticide are comminuted in a stirred ball mill with the addition of 10 parts by wt. of dispersing and wetting agents and 70 parts by wt. of water or an organic solvent to give a fine suspension of the active compound. On dilution in water, a stable suspension of the active compound results. The active compound content in the composition is 20 wt. %
vi) Water-dispersible and water-soluble granules (WG, SG)
   50 parts by wt. of the pesticide are finely ground with the addition of 50 parts by wt. of dispersing and wetting agents and are prepared as water-dispersible or water-soluble granules by means of industrial equipment (e.g. extrusion, spray tower, fluidized bed). On dilution in water, a stable dispersion or solution of the active compound results. The composition has an active compound content of 50 wt. %.
vii) Water-dispersible and water-soluble powders (WP, SP, SS, WS)
   75 parts by wt. of the pesticide are ground in a rotor-stator mill with the addition of 25 parts by wt. of dispersing and wetting agents and silica gel. On dilution in water, a stable dispersion or solution of the active compound results. The active compound content of the composition is 75 wt. %
viii) Gels (GF)
   20 parts by wt. of the pesticide, 10 parts by wt. of dispersing agent, 1 part by wt. of swelling agent (“gelling agent”) and 70 parts by wt. of water or an organic solvent are ground in a ball mill to give a fine suspension. On dilution with water, a stable suspension with an active compound content of 20 wt. % results.

2. Composition types for direct application
ix) Dusts (DP, DS)
   5 parts by wt. of the pesticide are finely ground and mixed intimately with 95 parts by wt. of finely divided kaolin. A dust composition with an active compound content of 5 wt. % is thereby obtained.
x) Granules (GR, FG, GG, MG)
   0.5 part by wt. of the pesticide are finely ground and bound with 99.5 parts by wt. of carrier substances. The usual processes here are extrusion, spray drying or fluidized bed. Granules for direct application with an active compound content of 0.5 wt. % are thereby obtained.
xi) ULV solution (UL)
   10 parts by wt. of the pesticide are dissolved with 90 parts by wt. of an organic solvent, e.g. xylene. A composition for direct application with an active compound content of 10 wt. % are thereby obtained.

The compounds can be used as such or in the form of their compositions, e.g. in the form of directly sprayable solutions, powders, suspensions, dispersions, emulsions, oil dispersions, pastes, dust compositions, scattering compositions or granules, by spraying, misting, dusting, scattering, laying out of baits, brushing, dipping or pouring. Aqueous use forms can be prepared from emulsion concentrates, pastes or wettable powders (spray powders, oil dispersions) by addition of water. For the preparation of emulsions, pastes or oil dispersions, the substances can be homogenized in water as such or as a solution in an oil or solvent, by means of wetting, adhesive, dispersing or emulsifying agents. However, concentrates comprising wetting, adhesive, dispersing or emulsifying agent and possibly solvent or oil which are suitable for dilution with water can also be prepared from the active substance.

The active compound concentrations in the ready-to-use formulations can be varied within relatively wide ranges. In general, they are between 0.0001 and 10%, preferably between 0.01 and 1%. The amounts applied for use in plant protection are between 0.01 and 2.0 kg of active compound per ha, depending on the nature of the desired effect. In the treatment of plant propagation materials, e.g. seed, in general active compound amounts of from 1 to 1,000 g/100 kg, preferably 5 to 100 g/100 kg of propagation material or seed are used. For use in the protection of material or stored products, the amount of active compound applied depends on the nature of the field of use and of the desired effect. Conventional amounts applied in the protection of materials are, for example, 0.001 g to 2 kg, preferably 0.005 to 1 kg of active compound per cubic meter of material treated.

The present invention furthermore relates to UV absorbers of the abovementioned formula I wherein AO represents CH₂CH₂NH if both R² and R³ both represent H. Preferably, UV represents a free radical chosen from the structures of the formulae B and C. Preferably, AO represents CH₂CH₂NH.

In a preferred embodiment, the invention relates to a UV absorber, wherein the UV absorber corresponds to a structure of the formula IX

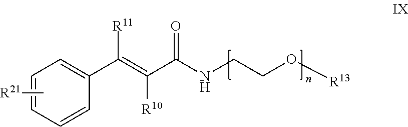

wherein n: 3 to 50, preferably 3 to 25; and R¹⁰, R¹¹, R¹² and R¹³ are as defined above.

In a further preferred embodiment, the invention relates to a UV absorber, wherein the UV absorber corresponds to a structure of the formula X

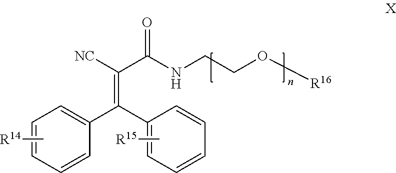

wherein n: 3 to 50, preferably 3 to 25; and R¹⁴, R¹⁵ and R¹⁶ are as defined above.

In a further preferred embodiment, the invention relates to a UV absorber, wherein the UV absorber corresponds to a structure of the formula XI

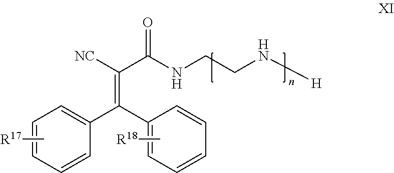

wherein n: 3 to 50, preferably 3 to 25; and R¹⁷ and R¹⁸ are as defined above.

In a further preferred embodiment, the invention relates to a UV absorber, wherein the UV absorber corresponds to a structure of the formula XII

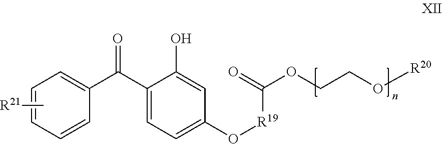

wherein n: 3 to 50, preferably 3 to 25, in particular 3 to 10; and R¹⁹, R²¹ and R²¹ are as defined above.

In a further preferred embodiment, the invention relates to a UV absorber, wherein the UV absorber corresponds to a structure of the formula XIII

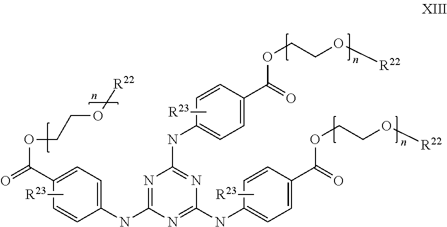

wherein n: 3 to 50, preferably 3 to 25, in particular 3 to 10; and R²² and R²³ are as defined above.

The UV absorbers according to the invention conventionally have a surface tension at the interface of water to air at 25° C. of at most 50 mN/m, preferably of at most 46 mN/m, particularly preferably at most 44 mN/m, specifically at most 40 mN/m.

The present invention furthermore relates to a use of the UV absorbers according to the invention in agrochemical compositions. Preference is given to the use in agrochemical compositions. Particular preference is given to the use for stabilizing UV-sensitive pesticides, particularly against sunlight. Preferred UV absorbers are those of the formulae III to XIV, in particular of the formulae IX to XIII. Suitable agrochemical compositions are as described above.

The present invention furthermore relates to a method of controlling phytopathogenic fungi and/or undesirable plant growth and/or undesirable insect or mite infestation and/or of regulating plant growth, wherein the composition according to the invention is allowed to act on the particular pests, their habitat or the plants to be protected from the particular pest, the soil and/or on the undesirable plants and/or the crop plants and/or their habitat.

An advantage of the present invention is that they stabilize UV-sensitive pesticides, in particular already at low concentrations of UV absorber. A further advantage is that fewer surfactants need to be employed in order to obtain a high stability of active compound dispersions, in particular active compound suspensions. The UV absorbers are readily soluble in agrochemical formulations, or very readily compatible with agrochemical formulations, such as aqueous emulsions and aqueous suspensions. For example, also no additional emulsifier is necessary in order to incorporate the UV absorbers into the formulation.

The following examples illustrate the invention without limiting it.

### EXAMPLES

Pluriol® A350E: Polyalkoxylene glycol monomethyl ether, OH number approx. 160 mg of KOH/g, molar mass approx. 350 g/mol, determined by means of OH number, commercially obtainable as Pluriol® A350E from BASF.
Pluriol® A500E: Methyl-polyethylene glycol, OH number approx. 110 mg of KOH/g, molar mass approx. 500 g/mol, determined by means of OH number, commercially obtainable as Pluriol® A500E from BASF SE.
Pluriol® A500PE: Butyl-polyethylene glycol/propylene glycol copolymer, OH number approx. 115 mg of KOH/g, molar mass approx. 500 g/mol, determined by means of OH number, commercially obtainable as Pluriol® A500PE from BASF SE.
Pluronic® 10500: Poly(ethylene glycol-block-propylene-block-ethylene glycol), with a propylene glycol block of a molar mass of 3,250 g/mol and a total molecular weight of 6,500 g/mol (commercially obtainable as Pluronic® PE 10500 from BASF SE).
Wettol D1: Sodium salt of a condensation product of phenolsulfonic acid-urea-formaldehyde (commercially obtainable as Wettol® D1 from BASF SE).
Bactericide: Aqueous mixture of 2.5 wt. % of 2-methyl-4-isothiazolin-3-one (MIT) and 2.5 wt. % of 1,2-benzisothiazolin-3-one (BIT) (commercially obtainable as Acticide® MBS from Thor)
Defoamer: Silicone-based, active content 20 wt. % (commercially obtainable as Silikon SRE-PFL from Wacker)
Xanthan: Granular xanthan gum, 14 wt. % water content, viscosity 2,000 mPas as a 0.3 wt. % solution by the Brookfield method (commercially obtainable as Rhodopol® G from Rhodia).
Lupasol® FG: A polyethyleneimine having a molar mass of about 800 g/mol (determined by means of light scattering), a ratio of primary to secondary to tertiary amine groups of 1 to 0.82 to 0.53 and a water content of less than 2 wt. %, commercially obtainable as Lupasol® FG from BASF SE.
iso-Tridecanol N: A branched aliphatic and primary C13 alcohol which was obtained by trimerization of butene and subsequent hydroformylation and hydrogenation (commercially obtainable as Tridecanol® N from BASF SE).
Tinuvin® 384-2: A UV absorber commercially obtainable from CIBA AG from the class of hydroxyphenylbenzotriazoles (95% benzenepropanoic acid 3-(2H-benzotriazol-2-yl)-5-(1,1-dimethylethyl)-4-hydroxy-C7-9-alkyl ester and 5% 1-methoxy-2-propyl acetate).
Uvinul® 3035: Ethyl 2-cyano-3,3-diphenylacrylate, compound having the structural formula (4), commercially obtainable as Uvinul® 3035 from BASF SE.
Uvinul® P25: p-Aminobenzoic acid ethoxylate (45) (molecular weight approx. 1,265 g/mol, the sum of x+y+z is about 25) is a commercially obtainable product with the name Uvinul® P25 from BASF SE.

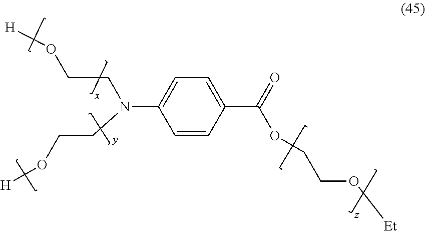

### Example 1A

### Synthesis of 2-cyano-3,3-diphenyl-acrylic acid (5)

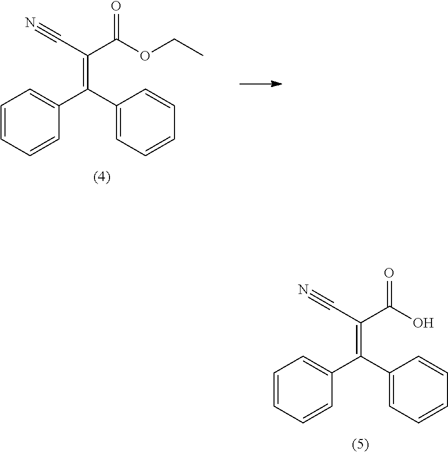

205 g (0.74 g) of Uvinol 3035 (4) were suspended in 2 I of 50% strength methanol, and 71.2 g (0.89 mol) of 50% strength sodium hydroxide solution were added. The mixture was stirred at 20° C. overnight. Thereafter, the mixture was rendered acidic with 102 g (0.89 mol) of 32% strength hydrochloric acid. The precipitate was filtered off and washed 2 times with 0.5 I of water each time. When dried, 170 g of a yellowish powder (5) were obtained (yield=92%).

### Example 1B

### Synthesis of 2-cyano-3,3-diphenyl-acryloyl chloride (6)

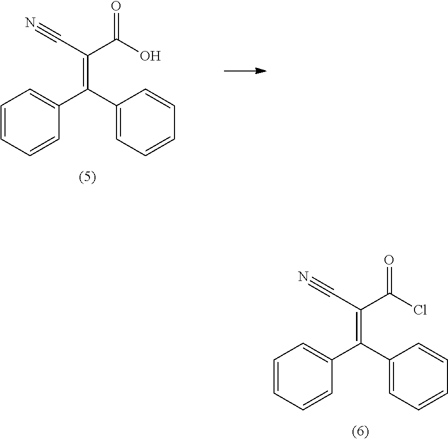

140 g (0.56 mol) of (5) were initially introduced into the reaction vessel with 3 drops of dimethylformamide in 0.5 I of methylene chloride at 20° C. 179 g (1.41 mol) of oxalyl chloride (1.41 mol) were added dropwise at 20° C. within 60 minutes. The mixture was stirred under reflux for 24 hours and then concentrated to dryness on a rotary evaporator. 149 g of a yellow solid (6) were obtained (yield=99%).

### Example 2A

### Synthesis of 2-cyano-3,3-diphenyl-acrylic acid [Pluriol A350E] ester (27)

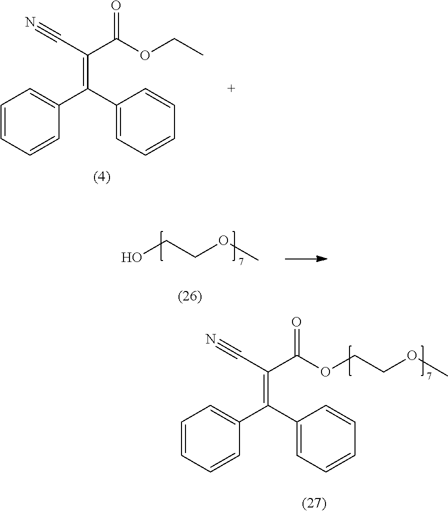

63.4 g (189 mmol) of Pluriol A350E (26) were stirred at 150° C. for 30 minutes, nitrogen being passed through by immersion during the entire reaction time. 50.0 g (180 mmol) of Uvinol 3035 (4) and 0.58 g (2 mmol) of titanium(IV) isopropoxide were then added. The reaction mixture was stirred at 155° C. for 24 hours. The ethanol formed was distilled off. The mixture was taken up in 200 ml of methylene chloride, 350 mg (3 mmol) of phosphoric acid 85% were added and the solution was left to stand at 20° C. for 24 hours. The product was purified by flash chromatography with 1 kg of silica gel 60. For this, the solution was introduced on to the flash column and the educt (4) was eluted from the column with methylene chloride, followed by the product (27) with methanol. The methanolic solution was concentrated to dryness on a rotary evaporator. 94 g of an orange viscous liquid (27) were obtained (yield=95%).

### Example 2B

### Synthesis of 2-cyano-3,3-diphenyl-acrylic acid [Pluriol A500E] ester (29)

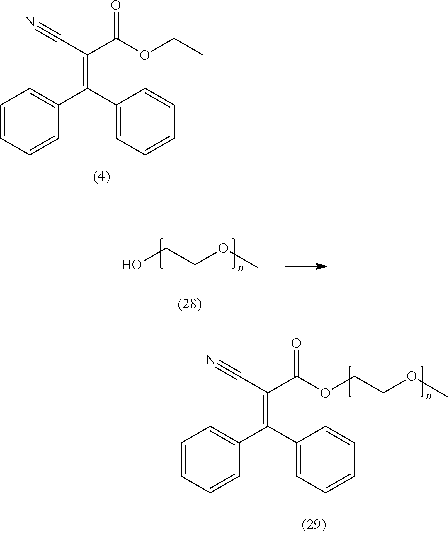

Nitrogen was passed through by immersion during the entire reaction time. 83.3 g (180 mmol) of Pluriol A500E (28) and 50.0 g of Uvinul 3035 (4) were initially introduced into the reaction vessel at 20° C. and the mixture was then stirred at 150° C. for 30 minutes. Thereafter, 0.58 g (2 mmol) of titanium(IV) isopropoxide was added. The reaction mixture was stirred at 155° C. for 24 hours. The ethanol formed was distilled off during the reaction. 118 g of a brown viscous liquid (29) were obtained (yield=95%).

### Example 2C

### Synthesis of 2-cyano-3,3-diphenylacrylic acid [Pluriol A500PE] ester (31)

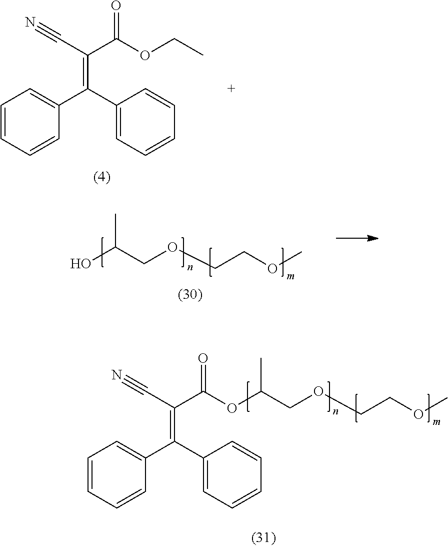

Nitrogen was passed through by immersion during the entire reaction time. 77.4 g (189 mmol) of Pluriol A500PE (30) and 50.0 g (180 mmol) of Uvinul 3035 (4) were initially introduced into the reaction vessel at 20° C. and the mixture was stirred at 155° C. for 30 minutes. 0.58 g (2 mmol) of titanium(IV) isopropoxide was then added and the reaction mixture was stirred at 155° C. for 21 hours. The ethanol formed was distilled off. 0.5 ml of water was added to the mixture and the mixture was stirred for 10 minutes and then taken up in 200 ml of methylene chloride. The product was purified by flash chromatography with 1 kg of silica gel 60. For this, the solution was introduced onto the flash column and the educt (4) was eluted from the column with methylene chloride, followed by the product (31) with methanol. The methanolic solution was concentrated to dryness on a rotary evaporator. 110 g of an orange viscous liquid (31) were obtained (yield=83%).

### Example 2D

### Synthesis of 2-cyano-3,3-diphenylacrylic acid [EO-tridecanol]ester (14)

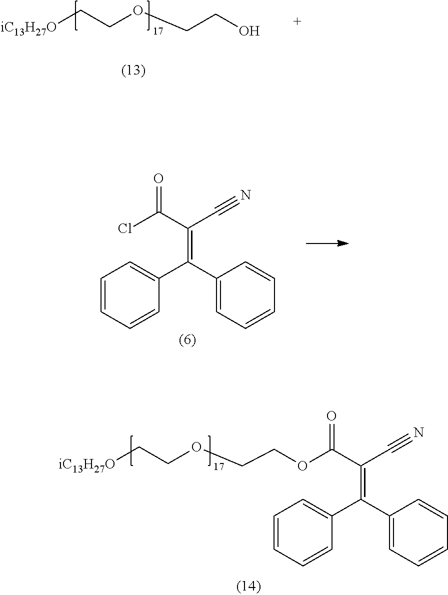

Powdered KOH (2 g, 0.036 mol) is added to iso-tridecanol (160 g, 0.8 mol) in a pressure autoclave and dewatering is then carried out at 95° C. under 20 mbar for 1 h. The mixture is then rendered inert with nitrogen and heated to 100° C. Ethylene oxide (634 g, 14.4 mol) is then metered in up to a maximum pressure of 6 bar in the course of 8 h and, when the addition has ended, the mixture is subsequently stirred for a further 3 h. Finally, synthetic magnesium silicate (3 wt. %) is added to the compound and the mixture is filtered. Tridecyloctadecaoxyethylene glycol (13) is obtained (794 g; OH number 69 mg of KOH/g). Compound (13) (49.6 g, 0.05 mol) is dissolved in toluene (100 ml), while stirring, and triethylamine (5.06 g, 0.05 mol) is added. A solution of compound (6) (13.4 g, 0.05 mol) in methylene chloride (50 ml) is added dropwise to this solution at 25° C. such that the temperature does not exceed 30° C. After stirring at 25° C. for 12 h, the solution is depleted in methylene chloride with the aid of a stream of N₂. The organic phase is then extracted by shaking three times with saturated NaCl solution, dried over sodium sulfate and freed from the solvent in vacuo. The target compound (14) is obtained in a yield of 58 g. The structure of (14) is confirmed by 1H-NMR spectroscopy.

### Example 3A

### Synthesis of (4-benzoyl-3-hydroxy-phenoxy)-acetic acid methyl ester (9)

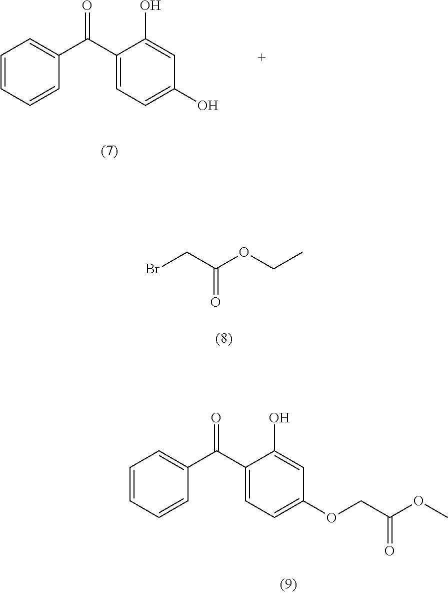

107 g (0.5 mol) of 2,4-dihydroxybenzophenone 99% (7) and 69 g (0.5 mol) of potassium carbonate were initially introduced into 0.65 I of methanol at 20° C. 98 g (0.5 mol) of bromoacetic acid ethyl ester (8) were added dropwise to this mixture at 20° C. in the course of 1 hour. The suspension was stirred under reflux for 24 h. The solid was filtered off and washed with 300 ml of methanol. The crude product was taken up in 1 I of methylene chloride, and 30 g of sodium sulfate and 15 g of active charcoal were then added. The mixture was stirred at 20° C. for 15 minutes and then filtered with suction over 100 g of silica gel 60. The solution was concentrated on a rotary evaporator. 70 g of white needles (9) were obtained (yield=49%).

### Example 3B

### Synthesis of (4-benzoyl-3-hydroxy-phenoxy)-acetic acid [Pluriol A350E] ester (34)

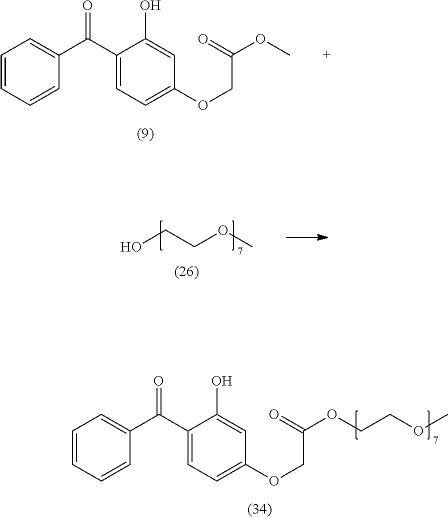

Nitrogen was passed through by immersion during the entire reaction time. 61.4 g (192 mmol) of Pluriol A350E (26) and 50.0 g (175 mmol) of (9) were initially introduced into the reaction vessel at RT and the mixture was then stirred at 155° C. for 30 minutes. 0.58 g (2 mmol) of titanium(IV) isopropoxide was then added and the reaction mixture was stirred at 155° C. for 18 hours. The methanol formed was distilled off. The mixture was taken up in 200 ml of methylene chloride, 350 mg (3 mmol) of phosphoric acid 85% were added and the solution was left to stand at 20° C. for 24 hours. The product was purified by flash chromatography with 1 kg of silica gel 60. For this, the solution was introduced onto the flash column and the educt (9) was eluted from the column with methylene chloride, followed by the product (34) with methanol. The methanolic solution was concentrated to dryness on a rotary evaporator. 99 g of an orange viscous liquid (34) were obtained (yield=99%).

### Example 3C

### Synthesis of (4-benzoyl-3-hydroxy-phenoxy)-acetic acid [Pluriol A500E] ester (35)

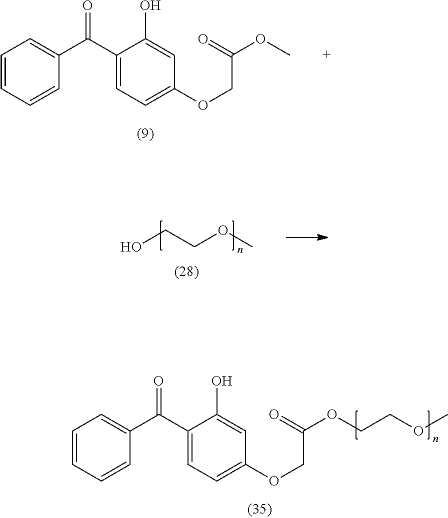

Nitrogen was passed through by immersion during the entire reaction time. 44.4 g (96.0 mmol) of Pluriol A500E (28) and 25.0 g (87.3 mmol) of (9) were mixed, and thereafter the mixture was stirred at 155° C. for 30 minutes. After addition of 0.29 g (1 mmol) of titanium(IV) isopropoxide, the reaction mixture was stirred at 155° C. for 20 hours. The methanol formed was distilled off. The mixture was taken up in 100 ml of methylene chloride. The product was purified by flash chromatography with 435 g of silica gel 60. For this, the solution was introduced on to the flash column and the educt (9) was eluted from the column with methylene chloride, followed by the product (35) with methanol. The methanolic solution was concentrated to dryness on a rotary evaporator. 54 g of an orange viscous liquid (35) were obtained (yield=86%).

### Example 4A

### Synthesis of 2,4,6-trianilino-p-(carboxy-ethyl)-1,3,5-triazine (12)

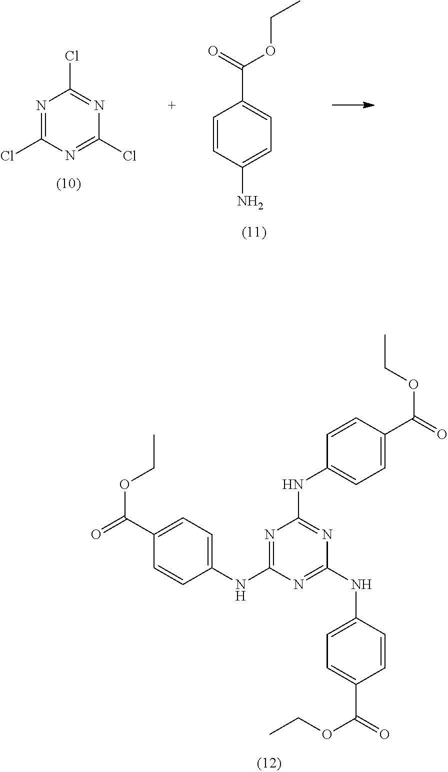

30.0 g (0.163 mol) of cyanuric chloride (10) and 33.7 g (0.244 mol) of potassium carbonate were initially introduced into 1 I of 1,4-dioxane under a nitrogen atmosphere at RT. 82.3 g (0.488 mol) of ethyl 4-aminobenzoate 98% (11) were added to this mixture in portions. The mixture was stirred under reflux for 1 hour. The mixture was filtered with suction and the suction filter cake was washed with 400 ml of 80° C. hot water and with 250 ml of cold water. After drying in a vacuum drying cabinet, 87 g of a white solid (12) were obtained (yield=94%).

### Example 4B

### Synthesis of 2,4,6-trianilino-p-(carboxy-[Pluriol A500E]-yl)-1,3,5-triazine (36)

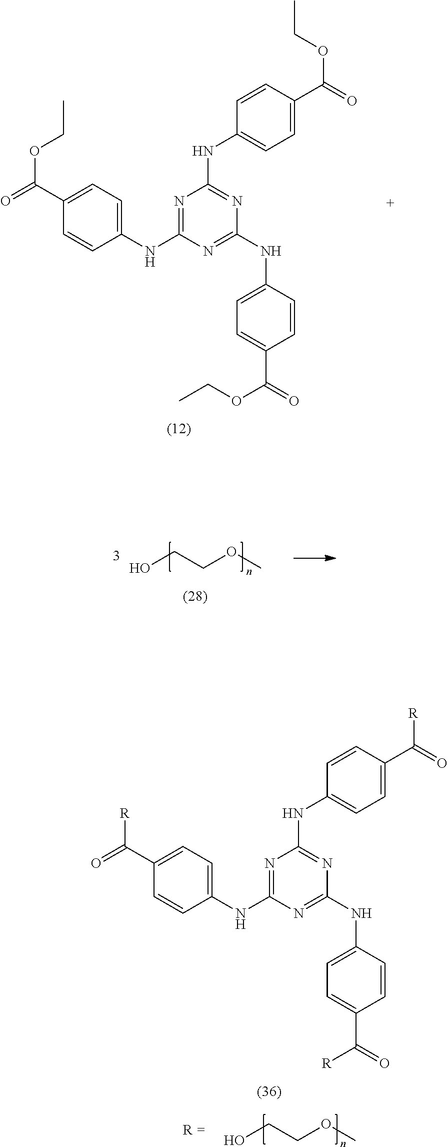

Nitrogen was passed through by immersion during the entire reaction time. 53.7 g (116 mmol) of Pluriol A500E (28) and 20.0 g (35.1 mmol) of (12) were mixed, and thereafter the mixture was stirred at 150° C. for 30 minutes. After addition of 0.85 g (3 mmol) of titanium(IV) isopropoxide, the reaction mixture was stirred at 155° C. for 17 hours. The ethanol formed was distilled off. 63 g of an orange viscous liquid (36) were obtained (yield=99%).

### Example 5A

### Synthesis of Uvinul 3035-Lupasol FG-amide (38)

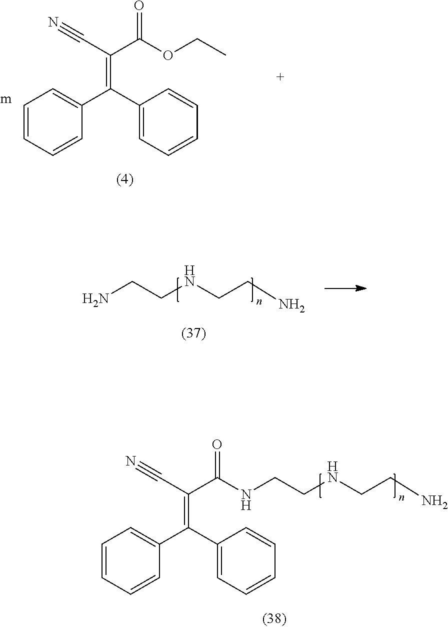

Nitrogen was passed through by immersion during the entire reaction time. 17 g (61.6 mmol; 5 mol %) of Uvinul 3035 (4) and 75 g (1.23 mol) of Lupasol FG (37) (amine number=16.34 mmol/g) were mixed at RT and the mixture was stirred at 110° C. for 2 hours. The ethanol formed was distilled off. 82 g of an amber-colored viscous liquid (38) were obtained.

### Example 5B

### Synthesis of Uvinul 3035-Lupasol FG-amide (39)

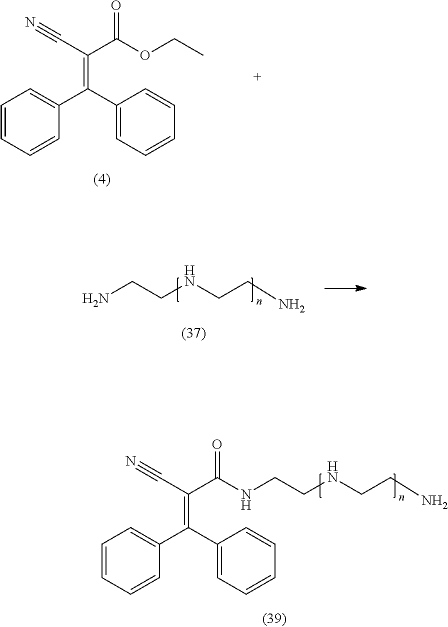

Nitrogen was passed through by immersion during the entire reaction time. 34 g (123 mmol; 10 mol %) of Uvinul 3035 (4) and 75 g (1.23 mol) of Lupasol FG (37) (AN=16.34 mmol/g) were mixed at 20° C. and the mixture was stirred at 110° C. for 2 hours. The ethanol formed was distilled off. 84 g of an amber-colored viscous liquid (39) were obtained.

### Example 6A

### Synthesis of 3,6,9,12-tetraoxatridecylcinnamic acid amide (42)

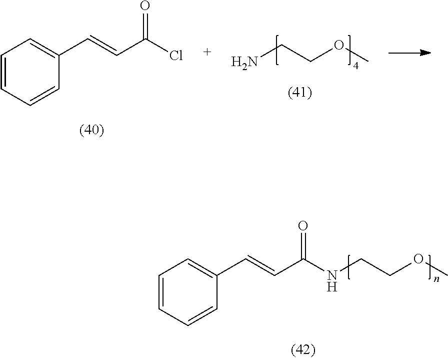

37.9 g (182 mmol) of (41) and 9.5 g (90 mmol) of sodium carbonate were initially introduced into 50 ml of methylene chloride at 0 to 5° C. A solution of 30 g (180 mmol) of (40) in 50 ml of methylene chloride was added dropwise to this mixture in the course of 1 hour. The mixture was stirred at 20° C. overnight. A further 100 ml of methylene chloride were added to the suspension and the mixture was then extracted by shaking 3 times with 200 ml of a hydrochloric acid saturated sodium chloride solution each time. The organic phase was dried with sodium sulfate and thereafter filtered and concentrated on a rotary evaporator at 50° C. 60 g of an amber-colored liquid (42) were obtained (yield=98%).

### Example 6B

### Synthesis of cinnamic acid 3,6,9,12-tetraoxatridecyl ester (44)

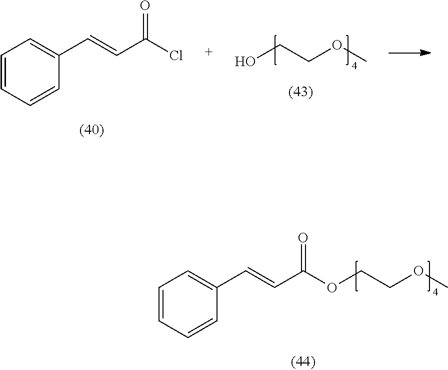

36.8 g (180 mmol) of (43) and 18.2 g (180 mmol) of triethylamine were initially introduced into 50 ml of methylene chloride at 20° C. A solution of 30 g (180 mmol) of (40) in 50 ml of methylene chloride was added dropwise to this mixture at RT in the course of 2 hours. The mixture was stirred at 20° C. overnight. The suspension was extracted by shaking 3 times with 75 ml of saturated sodium chloride solution each time. The organic phase was dried with sodium sulfate, and thereafter filtered and concentrated on a rotary evaporator at 50° C. 55 g of a yellowish partly crystalline liquid (44) were obtained (yield=89%).

### Example 7

### Investigation of the Action of the Stabilizers

A formulation comprising 100 g/l of metaflumizone and 40 g/l of UV absorber was prepared by two different methods:

a) Wet grinding with subsequent addition of UV absorber as in Example 9.

b) Wet grinding with UV absorber as in Example 8.

About 20 mg of the mixture prepared in this way were applied to a microscope slide, allowed to dry on for 30 minutes and exposed to light continuously for seven days (Atlas Suntest CRT plus, “Outdoor” setting, exposure to light corresponds to the same spectrum and intensity as normal sunlight at midday in summer). After the exposure time, the samples were dissolved in dimethylsulfoxide. The residual content of metaflumizone was determined by means of quantitative UPLC (column BEH C18 1.7 μm 2.1×100; elution with a gradient of acetonitrile/0.1% H₃PO₄ increasing from 5/95 to 95/5). For comparison, a sample without UV absorber was also allowed to run (reference) in each exposure series.

Evaluation: 2 samples were dissolved again as blank samples immediately after the drying on, without exposure to light, and the content of metaflumizone was determined. The resulting mean was set at 100% and the remaining samples were standardized against this. The results are summarized in Table 1:

TABLE 1

| Structure of the UV absorber | Formulation by | Residual content of |
|---|---|---|
| from Example | method | metaflumizone (%) |
| without UV absorber^{a)} | b) | 58.1 |
| 2A | a) | 87.1 |
| 2B | a) | 76.7 |
| 3B | b) | 82.8 |
| 3B | a) | 83.2 |
| 3B | a) | 85.9 |
| 4B | a) | 77.5 |
| 5A | a) | 67.2 |
| 5B | a) | 83.0 |
| 6A | a) | 74.7 |
| 6B | a) | 66.5 |
| Uvinul P25^{a)} | a) | 53.3 |

^{a)}not according to the invention

The data showed that due to the UV absorber according to the invention less pesticide was degraded than without UV absorber. It was moreover shown that if the UV absorber according to the invention is present, less pesticide is degraded than if Uvinul® P25 is present.

### Example 8

### Stability of Suspension Concentrate—Wet Grinding with UV Absorber

The suspension concentrates A1 to A6 and C1 were prepared by wet grinding (1 h, at 3,000 rpm, Dispermat) of the components mentioned in Table 2 and 105 g of metaflumizone (95 wt. % purity) and 70 g of 1,2-propylene glycol. A homogeneous stable suspension in which at least 90% of the solid particles had a particle size of less than 5 μm and D(4,3) was 1.0 μm was obtained. In each case 20 g of Wettol D1, 5.0 g of defoamer, 3.0 g of xanthan and 2.0 g of bactericide were added to these suspensions which were made up to 1.0 I with water. After storage at 54° C. for two weeks, no change in the particle size and the particle distribution was found.

A sample from all the experiments was in each case diluted with CIPAC water D (comprising 342 ppm of Ca/Mg ions) to a 0.1 and 1 wt. % strength suspension. Practically no sediments were found after storage for 6 h.

The experiment shows that the addition of UV absorbers according to the invention does not decrease the stability of suspension concentrates (SC) compared with SC without UV absorber. The stability of A1 and A2 comparable to that of A4 and A5 demonstrates that the SC formulation even manages with less dispersing agent, namely 111 g/l instead of 222 g/l.

TABLE 2

| Mixture | A1 | A2 | A4 | A5 | C1^{a)} |
|---|---|---|---|---|---|
| Pluronic 10500 [g/l] | 111 | 111 | 222 | 222 | 167 |
| UV absorber Example 2A [g/l] | 40 | — | 80 | — | — |
| UV absorber Example 3B [g/l] | — | 40 | — | 80 | — |

^{a)}not according to the invention

### Example 9

### Stability of Suspension Concentrate—Subsequent Addition of UV Absorber

The suspension concentrates A7 to A17 were prepared by wet grinding (1 h, at 3,000 rpm, Dispermat) of 105 g of metaflumizone (95 wt. % purity), 70 g of 1,2-propylene glycol and 111 g of Pluronic 10500. A homogeneous stable suspension in which at least 90% of the solid particles had a particle size of less than 5 pm and D(4,3) was 1.0 μm was obtained. In each case 40 g of UV absorber (see Table 3; except for in Experiment C1), 20 g of Wettol D1, 5.0 g of defoamer, 3.0 g of xanthan and 2.0 g of bactericide were added to these suspensions which were made up to 1.0 I with water. After storage at 54° C. for two weeks, no change in the particle size and the particle distribution of the active compound was found.

A sample from all the experiments was in each case diluted with CIPAC water D (comprising 342 ppm of Ca/Mg ions) to a 0.1 and 1 wt. % strength suspension of the active compound. Practically no sediments of the active compound were found after storage for 6 h.

TABLE 3

| | UV absorber |
|---|---|
| Experiment | from Example |
| C1^{a)} | none |
| A7 | 6A |
| A8 | 6B |
| A9 | 2A |
| A10 | 2B |
| A13 | 5A |
| A14 | 5B |
| A15 | 3B |
| A16 | 3C |
| A17 | 4B |

^{a)}not according to the invention, for the preparation see Example 9.

The experiment shows that the addition of UV absorbers according to the invention does not decrease the stability of suspension concentrates. The high stability was in turn achieved at a low concentration of dispersing agent (111 g/l), as in Example 8.

### Example 10

### Stability of Suspension Concentrate—Subsequent Addition of UV Absorber

The suspension concentrates A21 to A31 were prepared as in Example 10, except that the concentration of Pluronic 10500 was 222 g/l and that of UV absorber was 80 g/l (Table 4). After storage at 54° C. for two weeks, no change in the particle size and the particle distribution was found.

A sample from all the experiments was in each case diluted with CIPAC water D (comprising 342 ppm of Ca/Mg ions) to a 0.1 and 1 wt. % strength suspension. Practically no sediments were found after storage for 6 h.

TABLE 4

| | UV absorber |
|---|---|
| Experiment | from Example |
| C1^{a)} | none |
| A21 | 6A |
| A22 | 6B |
| A23 | 2A |
| A24 | 2B |
| A27 | 5A |
| A28 | 5B |
| A29 | 3B |
| A30 | 3C |
| A31 | 4B |

^{a)}not according to the invention, for the preparation see Example 9.

The experiment shows that the addition of UV absorbers according to the invention does not decrease the stability of the suspension concentration.

### Example 11

### Test of the UV Stability of Alpha-Cypermethrin

A solution of 20 mg of alpha-cypermethrin and 5 or 10 mg (i.e. 25 or 50 wt. % respectively with respect to the active compound) of UV absorber in acetone was prepared. A few drops were placed on a glass plate, allowed to dry for one hour and then irradiated with UV/VIS light of wavelength from 300 to 800 nm for 24 h. The irradiated mixture was then dissolved in tetrahydrofuran and the content of alpha-cypermethrin (i.e. recovery) was determined by means of HPLC chromatography. An identical sample which was stored in darkness for 24 h was investigated as a comparison. The experiment shows that the UV absorbers significantly increase the stability of UV-sensitive active compounds such as alpha-cypermethrin (Table 5).

TABLE 5

| | UV absorber | UV absorber | Recovery |
|---|---|---|---|
| Experiment | from Example | [wt. %] | [%] |
| A | — | 0 | 48 |
| B | — | 0 | 46 |
| C | 2A | 25 | 99 |
| D | 2B | 25 | 91 |
| E | 3B | 25 | 92 |
| F | 3C | 25 | 92 |
| G | 4B | 25 | 99 |
| H | 5A | 25 | 99 |
| I | 6A | 25 | 80 |
| J | 6B | 25 | 67 |
| K | 2A | 50 | 95 |
| L | 2B | 50 | 94 |
| M | 3B | 50 | 93 |
| N | 3C | 50 | 94 |
| O | 4B | 50 | 95 |
| P | 5A | 50 | 95 |

### Example 12

### Biological Tests with Metaflumizone

The plants (lima beans, Phaseolus lunatus) were irradiated with UV light constantly for 24 h per day at 26° C. in a growth chamber. The irradiation intensity in the UV range between 300 and 400 nm was measured and was 39 watt/m².

The plants were treated in the two-leaf stage with an aqueous spray liquor comprising the suspension concentrates (SC) A7 to A31 or C1 from Examples 9-11 with an application rate of 10 g/ha of metaflumizone. Seven and ten days after treatment (DAT) the active compound activity which remained was determined with a bioassay on larvae of Spodoptera eridania (southern armyworm). For this, the larvae were brought into contact with the plants and the mortality was determined three days later (Table 6). The experiment demonstrates that the insecticide is more stable to UV light in formulations comprising the UV absorbers tested than in formulations without UV absorber.

TABLE 6

| | |
|---|---|
| UV stabilizer | Mortality in [%] |

| SC | from Example | 7 DAT | 10 DAT |
|---|---|---|---|
| C1^{a)} | none | 55 | 22 |
| C2^{a)} | Uvinul P25 | 52 | 56 |
| A7 | 6A | 81 | 64 |
| A8 | 6B | 65 | 57 |
| A9 | 2A | 92 | 36 |
| A10 | 2B | 85 | 43 |
| A13 | 5A | 69 | 82 |
| A14 | 5B | 85 | 79 |
| A15 | 3B | 74 | 70 |
| A16 | 3C | 81 | 54 |
| A17 | 4B | 92 | 50 |
| A21 | 6A | 74 | 89 |
| A22 | 6B | 63 | 70 |
| A23 | 2A | 69 | 29 |
| A24 | 2B | 56 | 74 |
| A27 | 5A | 96 | 93 |
| A28 | 5B | 100 | 78 |
| A29 | 3B | 100 | 59 |
| A30 | 3C | 96 | 81 |
| A31 | 4B | 63 | 52 |

^{a)}Comparison experiment, not according to the invention

### Example 13

### Determination of the Surface Tension

The surface tension (ST) of the UV absorbers was determined at 25° C. at concentrations of the UV absorber of from about 1 mg/l to 5,000 mg/l. The ST of water to air is, for comparison, 72.4 mN/m, the ST of commercial surfactants is about 30-35 mN/m. The results are summarized in Table 7, which shows that the UV absorbers according to the invention can significantly lower the surface tension, i.e. that they are surface-active.

TABLE 7

| UV absorber from | |
|---|---|
| Example | ST [mN/m] |
| 2A | 43.1 |
| 3B | 45.7 |
| 2D | 33.9 |

1.-15. (canceled)

16. An agrochemical composition comprising a pesticide and a UV absorber of formula (I) 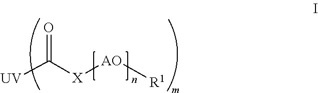
wherein
AO: C₂-C₄ alkoxy;
n: 3 to 50;
m: 1;
X: NH or O;
R¹: C₁-C₂₄ alkyl;
UV: is a group chosen from the formula A 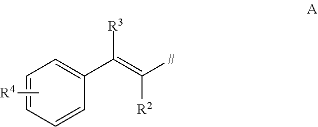
wherein R²: H, CN or CO₂—(C₁-C₁₆ alkyl);
R³: H, C₁-C₆ alkyl, phenyl or phenyl substituted by C₁-C₁₈ alkoxy;
R⁴: H or C₁-C₁₈ alkoxy; or formula B 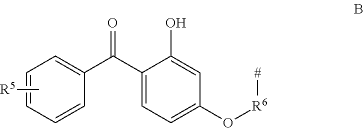
wherein R⁵: H, C₁-C₁₈ alkyl, OH or C₁-C₁₈ alkoxy;
R⁶: C₁-C₈ alkylene.

17. An agrochemical composition comprising pesticide and UV absorber,
wherein the UV absorber corresponds to the formula I 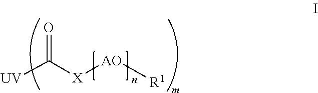
where AO: CH₂CH₂NH;
n: 3 to 50;
m: 1 if UV represents A or B, 3 if UV represents C;
X: NH or O;
R¹: H or C₁-C₂₄ alkyl;
UV: is a group chosen from the formulae A to C: 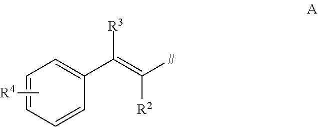
wherein R²: H, CN or CO₂—(C₁-C₁₆ alkyl);
R³: H, C₁-C₆ alkyl, phenyl or phenyl substituted by C₁-C₁₈ alkoxy;
R⁴: H or C₁-C₁₈ alkoxy; 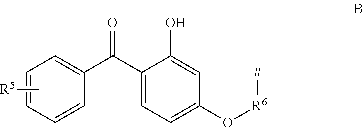
wherein R⁵: H, C₁-C₁₈ alkyl, OH or C₁-C₁₈ alkoxy;
R⁶: C₁-C₈ alkylene; or 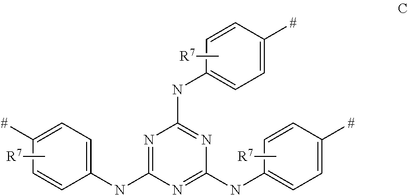
wherein R⁷: H or C₁-C₁₂ alkyl;
which are bonded via # to the carbonyl group of I.

18. The composition as claimed in claim 19, wherein the pesticide is UV-sensitive.

19. The composition as claimed in claim 16, wherein the weight ratio of pesticide to UV absorber is from 30:1 to 1:2.

20. The composition as claimed in claim 19, wherein the pesticide comprises
a) a herbicides selected from napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norflurazon, thiazopyr, triclopyr, dithiopyr, diflufenican, picolinafen, amidosulfuron, molinate, vernolate, prometon, metribuzin, azafenidin, carfentrazone-ethyl, sulfentrazone, metoxuron, monolinuron, fluchloralin and flurenol; or
b) a fungicides selected from cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichlofluamid, bromuconazole and myclobutanil; or
c) insectides selected from acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon.

21. The composition as claimed in claim 19, wherein the pesticide is alpha-cypermethrin or metaflumizone.

22. The composition as claimed in claim 19, wherein the UV absorber has a surface tension at the interface of water to air at 25° C. of at most 50 mN/m.

23. The composition as claimed in claim 19, comprising from 0.1% to 50% by weight of UV absorber, based on the composition.

24. The composition as claimed in claim 19, wherein n represents 3 to 25.

25. AUV absorber of the formula I as claimed in claim 17, wherein both R² and R³ represent H.

26. The UV absorber as claimed in claim 25, wherein UV is B or C.

27. The UV absorber as claimed in claim 25, wherein the polyethyleneimine group CH₂CH₂NH is branched.

28. The UV absorber as claimed in claim 25, wherein the molar ratio of primary/secondary amino groups of the polyethyleneimine group CH₂CH₂NH is in the range of from 1/0.5 to 1/1. 9.

29. A method of controlling phytopathogenic fungi and/or undesirable plant growth and/or undesirable insect or mite infestation and/or of regulating plant growth, wherein the composition as claimed in claim 16 is allowed to act on the particular pests, their habitat or the plants to be protected from the particular pest, the soil and/or on the undesirable plants and/or the crop plants and/or their habitat.

30. The method of claim 29, wherein the weight ratio of pesticide to UV absorber is from 30:1 to 1:2.

31. The method of claim 30, wherein the pesticide is UV-sensitive.

32. The method of claim 30, wherein the pesticide comprises
a) a herbicides selected from napropamide, propanil, bentazone, paraquat dichloride, cycloxydim, sethoxydim, ethalfluralin, oryzalin, pendimethalin, trifluralin, acifluren, aclonifen, fomesafen, oxyfluoren, ioxynil, imazetapyr, imazaquin, chloridazon, norflurazon, thiazopyr, triclopyr, dithiopyr, diflufenican, picolinafen, amidosulfuron, molinate, vernolate, prometon, metribuzin, azafenidin, carfentrazone-ethyl, sulfentrazone, metoxuron, monolinuron, fluchloralin and flurenol; or
b) a fungicides selected from cyprodinil, fuberidazole, dimethomorph, procloraz, triflumizole, tridemorph, edifenfos, fenarimol, nuarimol, ethirimol, quinoxylen, dithianon, metominostrobin, trifloxystrobin, dichlofluamid, bromuconazole and myclobutanil; or
c) insectides selected from acephate, azinphos-ethyl, azinphos-methyl, isofenphos, chlorpyriphos-methyl, dimethylvinphos, phorate, phoxim, prothiofos, cyhexatin, alanycarb, ethiofencarb, pirimicarb, thiodicarb, fipronil, bioallethrin, bioresmethin, deltamethrin, fenpropathin, flucythrinate, taufluvalinate, alpha-cypermethrin, metaflumizone, zeta-cypermethrin, resmethin, tefluthrin, lambda cyhalothrin and hydramethylnon.

33. The method of claim 30, wherein the pesticide is alpha-cypermethrin or metaflumizone.

33. The method of claim 30, wherein the UV absorber has a surface tension at the interface of water to air at 25° C. of at most 50 mN/m.

34. The method of claim 30, comprising from 0.1% to 50% by weight of UV absorber, based on the composition.

35. The method of claim 30, wherein n represents 3 to 25.
